(19) United States
(12) Patent Application Publication
(10) Pub. No.: US 2013/0261096 A1
(43) Pub. Date: Oct. 3, 2013

(54) Formulation For Hormonotherapy

(71) Applicant: Commissariat a I'energie Atomique et aux Energies Alternatives, Paris (FR)

(72) Inventors: Juliette MERIAN, Brive (FR); Raphaël BOISGARD, Nozay (FR); Bertrand TAVITIAN, Paris (FR); Isabelle TEXIER-NOGUES, Grenoble (FR)

(21) Appl. No.: 13/853,652

(22) Filed: Mar. 29, 2013

(30) Foreign Application Priority Data
Mar. 30, 2012 (FR) 12 52919

(51) Int. Cl. A61K 9/107; A61K 31/567; A61K 47/18; A61K 31/565

(52) U.S. Cl. CPC A61K 9/1075; A61K 31/565; A61K 31/567; A61K 47/18
USPC 514/182

(57) ABSTRACT

The present invention relates to a formulation as a nanoemulsion, comprising a continuous aqueous phase and at least one dispersed phase, and comprising:
an amphiphilic lipid,
a solubilizing lipid,
a hormone, a hormone agonist or antagonist or a mixture thereof, and
a co-surfactant comprising at least one chain consisting of alkylene oxide units for its use for hormonal treatment selected from contraception, reproduction assistance, post-menopausal hormonal treatment, treatment of menorrhagias, treatment of acne and treatment of adrenal dysregulations.

The present invention relates to the use of a formulation of the nanoemulsion type for hormonotherapy.

The delivery systems for hormonotherapy are described in the literature, with various (oral, transdermal, intra-uterine or intramuscular) administration routes and various (tablets, gels, emulsions, patch, implants, spray, vaginal ring . . . ) administration forms (Yoo et al., Journal of Controlled Release, 112, 2006, 1-14).

A few examples of formulations in the form of a suspension have been described. For example, Salem (International Journal of Nanomedecine, 2010, 5, 943-954) reports the use of a gel comprising a nanosuspension, the nanoparticles of which comprise progesterone, Pluronic F127 and steric acid, which may be administered via an intramuscular route and allowing limitation of the administration frequency. Mittal et al. (Journal of Pharmaceutical Sciences, 98(10), 2009, 3730-3734) describes polymeric nanoparticles (based on poly(lactate-co-glycolide)) comprising estradiol for oral administration allowing improvement in the bioavailability of the estradiol.

The oral route remains the most used administration route for hormonal treatment. Now, as the bioavailability of the hormones is low (mainly because of a first very significant hepatic passage and of metabolization of the hormone in the intestine, the administered doses of hormones are high, which causes significant secondary effects. The patches were developed as an alternative administration route, but the constancy of the delivery and the problems of penetration of the hormone remain challenges.

Moreover, patent application WO 2010/018223 describes the use of a formulation as a nanoemulsion, comprising a continuous aqueous phase and at least one dispersed phase comprising an amphiphilic liquid, a solubilizing lipid, a therapeutic agent and a co-surfactant comprising at least one chain consisting of alkylene oxide units, for delivering a therapeutic agent. This application by no means suggests that this formulation is targeting steroidal organs, and its use for hormonal treatment is not described.

The development of novel hormonal treatment methods is necessary, allowing minimization of the secondary effects and having good effectiveness, good tolerance by the patients and good bioavailability of the active ingredients.

For this purpose, the present invention provides a formulation comprising a hormone, a hormone agonist or a antagonist, and preferentially targeting steroidal organs, and which is consequently used for hormonotherapy.

### DEFINITIONS

In the sense of the present application, the expressions of <<hormonotherapy>> and <<hormonal treatment>> have the same meaning and mean administration of a natural or artificial hormone, whether this hormone is inhibitory of another hormone (administration of a hormone antagonist) or compensatory (administration of a hormone agonist for compensating a secretion deficiency for substutional hormonotherapy).

Within the scope of this discussion, by the term of <<nanoemulsion>> is meant a composition having at least two phases, generally an oily phase and an aqueous phase, wherein the average size of the dispersed phase is less than 1 micron, preferably from 10 to 500 nm and in particular from 20 to 100 nm, and most preferentially from 20 to 70 nm (see the article C. Solans, P. Izquierdo, J. Nolla, N. Azemar and M. J. Garcia-Celma, Curr. Opin. Colloid. In., 2005, 10, 102-110).

In the sense of the present application, the expression of <<dispersed phase>> designates the droplets comprising the optional oil/solubilizing lipid/amphiphilic lipid/co-surfactant/the hormone, the hormone agonist or antagonist or the mixture thereof. The dispersed phase is generally free of any aqueous phase.

The term of <<droplet>> encompasses both liquid oil droplets strictly speaking as well as solid particles stemming from emulsions of the oil-in-water type in which the oily phase is solid. In the latter case, this is often also referred to as a solid emulsion.

The term of <<lipid>> designates within the scope of this discussion, the whole of the fats or substances containing fatty acids present in the fats of animal origin and in vegetable oils. These are hydrophobic or amphiphilic molecules mainly consisting of carbon, hydrogen and oxygen and having a density below that of water, lipids may be in the solid state at room temperature (25° C.), like in waxes, or in the liquid state like in oils.

The term of <<phospholipid>> aims at lipids having a phosphate group, notably phosphoglycerides. Most often, phospholipids include a hydrophilic end formed by the optionally substituted phosphate group and two hydrophobic ends formed by chains of fatty acids. Among phospholipids, mention will be made in particular of phosphatylcholine, phosphatidyl ethanolamine, phosphatidyl inositol, phosphatidyl serine and sphingomyelin.

The term of <<lecithin>> designates phosphatidylcholine, i.e. a lipid formed from a choline, a phosphate, a glycerol and from two fatty acids. It more widely covers phospholipids extracted from living material, of vegetable or animal origin, insofar that they in majority consist of phosphatidylcholine. These lecithins generally form mixtures of lecithins bearing different fatty acids.

The term of <<fatty acid>> is meant to designate aliphatic carboxylic acids having a carbonaceous chain of at least 4 carbon atoms. Natural fatty acids have a carbonaceous chain from 4 to 28 carbon atoms (generally an even number). One refers to a fatty acid with a long chain for a length from 14 to 22 carbons and with a very long chain if there are more than 22 carbons.

By the term of <<surfactant>> is meant compounds with an amphiphilic structure which gives them particular affinity for interfaces of the oil/water and water/oil type, which gives them the capability of lowering the free energy of these interfaces and of stabilizing dispersed systems.

By the term of <<co-surfactant>> is meant a surfactant acting in addition to a surfactant, for further lowering the interface energy.

The term of <<hydrocarbon chain>> is meant to designate a chain consisting of carbon and hydrogen atoms, either saturated or unsaturated (double or triple bond). Preferred hydrocarbon chains are alkyls or alkenyls.

The term of <<alkylene>> is meant to designate a linear or branched, preferably linear, saturated hydrocarbon aliphatic divalent group.

By <<activated ester>>, is meant a group of formula —CO-LG, and by <<activated carbonate>>, is meant a group of formula —O—CO-LG wherein LG is a good leaving group notably selected from a chlorine, an imidazolyl, a pentafluorophenolate, a pentachlorophenolate, a 2,4,5-trichlorophenolate, a 2,4,6-trichlorophenolate, an —O-succinimidyl group, an —O-benzotriazolyl, —O-(7-aza-benzotriazolyl) and an —O-(4-nitrophenyl) group.

[Formulation]

According to a first object, the invention relates to a formulation as a nanoemulsion, comprising a continuous aqueous phase and at least one dispersed phase, and comprising:
an amphiphilic lipid,
a solubilizing lipid,
a hormone, a hormone agonist or antagonist or a mixture thereof, and
a co-surfactant comprising at least one chain consisting of alkylene oxide units,
   for its use for hormonal treatment, also called hormonotherapy.

The emulsion therefore is an emulsion of the oil-in-water type. It may be simple or multiple, notably by including in the dispersed phase, a second aqueous phase. Preferably it is simple.

Contraception, assisted reproduction, post-menopausal hormone treatment (also called hormonal replacement treatment (HRT)), treatment of menorrhagias, treatment of acne, and treatment of adrenal disregulations such as Addison's disease, adrenal fatigue and infertility are the most common hormonal treatments.

The invention also relates to the use of the formulation above for contraception, post-menopausal hormone treatment or assisted reproduction.

The invention is based on the unexpected discovery that the formulation described above specifically targets steroidal organs such as ovaries and adrenal glands. Therefore, the formulation is particularly suitable for use in hormonotherapy, where the delivery of a hormone, hormone agonist or antagonist is desired at these organs.

The formulation used also has other advantages which makes it interesting for its therapeutic use.

First of all, the zeta potential of the dispersed phase is less than 20 mV in absolute value, i.e. comprised between −20 and 20 mV. A high zeta potential in absolute value of the droplets has the effect of accumulation for certain organs, notably in the liver, spleen, lungs, in addition to kidneys. The formulation used is therefore particularly suitable for being administered into the body since it does not accumulate in these organs.

As explained above, it advantageously has good bioavailability and may give the possibility of limiting or even avoiding the secondary effects related to the administration of hormone, of hormone agonist or antagonist, generally observed with other delivery systems.

Further, the formulation used in the invention is advantageously stable: it may be stored for more than three months without observing any degradation.

Hormone and Hormone Agonist or Antagonist

A hormone treatment consists of administering a hormone, a hormone agonist or antagonist, which allows modulation of the activity of a receptor of a hormone, either by activating it (for an agonist) or by inhibiting it (for antagonist). For example, for estrogen hormone, as an agonist, mention may be made of regulators of tissue estrogenic activity (selective tissue estrogenic activity regulators (STEARs)), like tibolone, and as an antagonist, the selective modulators of estrogen receptors (selective estrogen receptor modulators (SERMs)), such as clomifene citrate, tamoxifene, raloxifene, toremifene or lasofoxifene.

Typically, in the aforementioned formulation:

the hormone is selected from:
the estrogen, notably selected from estrone, estriol and estradiol (17-β-estradiol),
   progesterone,
   hydrocortisone,
   5-dehydroepiandrosterone (DHEA).
   gonadotropins, and
   kisspeptin,
the hormone agonist is:
   an estrogen agonist, such as tibolone or an estrogen derivative selected from estrone, estriol and estradial esters (for example estradiol acetate or valerate), estropipate, estradiol cypionate, conjugate equine estrogens (CEE) and estradiol ethinyl, or
   a progestin (agonist of progesterone), such as medroxyprogesterone acetate, norethisterone, norethisterone acetate, lynestenol, levonorgestrel, norgestrel, norgestimate, desogestrel, etonogestrel, gestodene, norgestimate, norelgestromin, nandralone, 17-hydroxyprogesterone or drospirenone,
the hormone antagonist is:
   an antiandrogen, such as cyproterone acetate, or
   an antagonist of estrogens, such as clomifene citrate, tamoxifene, raloxifene, toremifene or lasofoxifene.

The aforementioned agonist and antagonist hormones are available commercially or may be synthesized with methods known to one skilled in the art.

Of course, the hormone, the agonist and/or antagonist of the formulation may be directly formulated in its active form or in the form of a prodrug.

The embodiments of the following Table 1 are preferred:

TABLE 1
Hormone, hormone agonist or antagonist or preferred mixture
thereof depending on the type of hormonotherapy.

| | |
|---|---|
| Hormonotherapy type | Hormone, hormone agonist or antagonist |
| Contraception | the estrogen or an estrogen agonist, |
| | preferably tibolone or an estrogene derivative selected from |
| | estrone, estriol and estradiol esters, estropipate, estradiol |
| | cypionate, conjugate equine estrogens (CEE) and ethinyl |
| | estradiol, |
| | preferably tibolone or ethinyl estradiol, |
| | preferably ethinyl estradiol, and/or |
| | progesterone or a progestin |
| Treatment of acne | an antiandrogen such as cyproterone acetate, and/or a |
| | progestin, such as medroxyprogesterone acetate, |
| | drospirenone or norgestimate |
| Treatment of menorrhagias | Progesterone or a progestin |
| Treatment of infertility due to | Tamoxifene, clomifene citrate, gonadotropins, kisspeptin |
| adrenal dysregulation or | |
| assisted reproduction | |
| Treatment of Addison's | hydrocortisone |
| disease | |
| Treatment of adrenal fatigue | hydrocortisone or 5-dehydroepiandrosterone, and optionally |
| | pantothenic acid and/or phosphatidylserine |
| Post-menopausal hormone | the estrogen or an estrogen antagonist, such as tibolone or |
| treatment (also called a | an estrogen derivative selected from estrone, estriol and |
| hormone replacement | estradiol esters, estropipate, estradiol cypionate, conjugate |
| treatment (HRT)) | equine estrogens (CEE) and ethinyl estradiol, |
| | preferably tibolone or ethinyl estradiol, |
| | preferably ethinyl estradiol, and |
| | optionally progesterone or a progestin. |

The formulation used most often comprises an amount from 0.001 to 30% by weight, generally less than 10% by weight, notably less than 3% by weight, preferably between 0.005 and 1% by weight of hormone, hormone agonist or antagonist or a mixture thereof. Indeed, these proportions are suitable for hormonotherapy and for these proportions, the size of the droplets is generally more homogeneous, which allows a more homogeneous release of the hormone, of the hormone agonist or antagonist or of the mixture thereof.

It is sometimes sought to formulate the nanoemulsion used with a maximum concentration of hormone, hormone agonist or antagonist, notably when they are not very soluble, in order to limit the volume and/or the duration of administration to the patient.

The formulation used in the invention has the advantage of having very good bioavailability (passes well through the barriers), this bioavailability being generally improved relatively to the existing delivery systems. Thus, more hormone or more hormone agonist or antagonist reaches the target, and it is possible to limit administered doses of hormone or hormone agonist or antagonist, which is advantageous not only in terms of costs, but especially since this allows limitation of the secondary effects generally observed upon administering the hormone, the hormone agonist or the hormone antagonist (as an example, estrogens administered at a high dose promote breast cancers in women during post-menopausal treatments).

Generally, the hormone, the hormone agonist and/or antagonist is lipophilic and is located in the dispersed phase; it is thus encapsulated in droplets.

Advantageously, the formulation used in the invention protects the hormone, the hormone agonist and/or antagonist which is located in the droplets, from the body of the patient to which the formulation is administered (notably by avoiding its premature metabolism). This protection also advantageously allows limitation of the secondary effects generally observed upon administering the hormone, the hormone agonist or the hormone antagonist.

Amphiphilic Lipid

The formulation used in the invention comprises at least one amphiphilic lipid which is located in the dispersed phase of the nanoemulsion.

In order to form a stable nanoemulsion, it is generally necessary to include in the composition at least one amphiphilic lipid as a surfactant. The amphiphilicity of the surfactant ensures stabilization of the oil droplets within the continuous aqueous phase.

Amphiphilic lipids include a hydrophilic portion and a lipophilic portion. They are generally selected from compounds for which the lipophilic portion comprises a linear or branched, saturated or unsaturated chain, having from 8 to 30 carbon atoms. They may be selected from phospholipids, cholesterols, lysolipids, sphingomyelins, tocopherols, glucolipids, stearylamines, cardiolipins of natural or synthetic origin; molecules consisting of a fatty acid coupled with a hydrophilic group through an ether or ester function such as sorbitan esters like for example sorbitan mono-oleates and mono-laurates sold under the names of Span® by Sigma;

polymerized lipids; conjugate lipids to short chains of polyethylene oxide (PEG) such as the non-ionic surfactants sold under the trade names of Tween® by ICI Americas, Inc. and Triton® by Union Carbide Corp; sugar esters such as saccharose mono- and di-laurate, mono- and di-palmitate, mono- and di-stearate; said surfactants may be used alone or as mixtures.

Phospholipids are the preferred amphiphilic lipids.

Lecithin is the more preferred amphiphilic lipid.

Generally, the dispersed phase will include from 0.001 to 99% by weight, preferably from 5 to 75% by weight, in particular from 5 to 50% and most particularly from 8 to 30% by weight of amphiphilic lipid.

The amount of amphiphilic lipid advantageously contributes to controlling the size of the dispersed phase of the nanoemulsion.

Solubilizing Lipid

The formulation used in the invention moreover comprises a solubilizing lipid, which is located in the dispersed phase of the nanoemulsion. This compound has the main mission of solubilizing the amphiphilic lipid, not very soluble in the oily phase of the nanoemulsion.

The solubilizing lipid is a lipid having sufficient affinity with the amphiphlic lipid for allowing its solubilization. Preferably, the solubilizing lipid is solid at room temperature.

In the case when the amphiphilic agent is a phospholipid, these may notably be derivatives of glycerol, and in particular glycerides obtained by esterification of glycerol with fatty acids.

The solubilizing lipid used is advantageously selected depending on the amphiphilic lipid used. It will generally have a close chemical structure, in order to ensure the sought solubilization. This may be an oil or a wax. Preferably, the solubilizing lipid is solid at room temperature (20° C.), but liquid at the temperature of the body (37° C.).

The preferred solubilizing lipids, in particular for phospholipids, are glycerides of fatty acids, notably of saturated fatty acids, and in particular of saturated fatty acids including 8 to 18 carbon atoms, still preferably 12 to 18 carbon atoms. Advantageously, this is a mixture of different glycerides.

Preferably, these are glycerides of saturated fatty acids including at least 10% by weight of C12 fatty acids, at least 5% by weight of C14 fatty acids, at least 5% by weight of C16 fatty acids and at least 5% by weight of C18 fatty acids.

Preferably, these are glycerides of saturated fatty acids including 0% to 20% by weight of C8 fatty acids, 0% to 20% by weight of C10 fatty acids, 10% to 70% by weight of C12 fatty acids, 5% to 30% by weight of C14 fatty acids, 5% to 30% by weight of C16 fatty acids and 5% to 30% by weight of C18 fatty acids.

Mixtures of semi-synthetic glycerides, solid at room temperature sold under the trade name Suppocire®NC by Gattefossé and approved for injection in humans are particularly preferred. The Suppocire®s of type N are obtained by direct esterification of fatty acids and of glycerol. These are hemi-synthetic glycerides of saturated C8-C18 fatty acids, the qualitative-quantitative composition of which is indicated in the table below.

The aforementioned solubilizing lipids give the possibility of obtaining a formulation in the form of an advantageously stable nanoemulsion. Without intending to be bound to a particular theory, it is assumed that the aforementioned solubilizing lipids give the possibility of obtaining droplets in the nanoemulsion having an amorphous core. The thereby obtained core has a high internal viscosity without however having any crystallinity. Now, crystallization is detrimental for the stability of the nanoemulsion since it generally leads to aggregation of the droplets and/or to expulsion of the encapsulated molecules outside the droplets. These physical properties promote physical stability of the nanoemulsion and the stability of the encapsulation over time of the hormone, of the hormone antagonist or agonist or of their mixture.

The amount of solubilizing lipid may widely vary depending on the nature and on the amount of amphiphilic lipid present in the oily phase. Generally, the oily phase will include from 1 to 99% by weight, preferably from 5 to 80% by weight and most particularly from 40 to 75% by weight of solubilizing lipid.

Composition of fatty acids for Suppocire ® NC from Gattefossé

| Chain length | [% by weight] |
|---|---|
| C8 | 0.1 to 0.9 |
| C10 | 0.1 to 0.9 |
| C12 | 25 to 50 |
| C14 | 10 to 24.9 |
| C16 | 10 to 24.9 |
| C18 | 10 to 24.9 |

Oil

The oily phase may moreover include one or several other oils, which are located in the dispersed phase.

The oils used preferably have a hydrophilic-lipophilic balance (HLB) of less than 8 and even more preferentially comprised between 3 and 6. Advantageously, the oils are used without any chemical or physical modification prior to formation of the emulsion.

The oils are generally selected from biocompatible oils, and in particular from oils of natural origin (vegetable or animal origin) or synthetic oils. Among such oils, mention may notably be made of oils of a natural vegetable origin among which notably appear soya bean, flax, palm, groundnut, olive, grape pip and sunflower oils; synthetic oils among which triglycerides, di-glycerides and mono-glycerides notably appear. These oils may be from first expressions, refined or inter-esterified.

The preferred oils are soya bean oil and flax oil.

Generally, if it is present, the oil will be contained in the oily phase in a proportion ranging from 1 to 80% by weight, preferably between 5 and 50% by weight and most particularly 10 to 30% by weight.

The oily phase may further contain other additives such as coloring agents, stabilizers, preservatives or other active ingredients, in a suitable amount.

Co-Surfactant

The formulation used in the invention usually comprises a co-surfactant which allows stabilization of the nanoemulsion.

The co-surfactant may moreover have other effects in the contemplated application of the nanoemulsion.

The co-surfactants which may used in the formulations used in the invention, are preferably water-soluble surfactant. Water-soluble surfactants are preferably alkoxylated (they comprise at least one chain consisting of alkylene oxide units) and preferably include at least one chain consisting of ethylene oxide units (PEO or PEG) or ethylene oxide and propylene oxide units. Preferably, the number of units in the chain varies between 2 and 500.

As an example of co-surfactants, mention may in particular be made of conjugate polyethylene glycol/phosphatidyl-ethanolamine (PEG-PE) compounds, fatty acid and polyethylene glycol ethers such as the products sold under the trade names of Brij® (for example Brij® 35, 58, 78 or 98) by ICI Americas Inc., fatty acid and polyethylene glycol esters such as the products sold under the trade names Myrj® by ICI Americas Inc. (for example Myrj® 45, 52, 53 or 59) and block copolymers of ethylene oxide and propylene oxide such as the products sold under the trade names Pluronic® by BASF AG (for example Pluronic® F68, F127, L64, L61, 10R4, 17R2, 17R4, 25R2 or 25R4) or products sold under the trade name Synperonic® by Unichema Chemie BV (for example Synperonic® PE/F68, PE/L61 or PE/L64).

Thus, the co-surfactant is located both in the continuous aqueous phase and in the dispersed phase. Indeed, the hydrophobic portion of the co-surfactant is inserted into the droplets of the dispersed phase, while the polyalkoxylated chains are in the continuous aqueous phase. In the present application, the described mass percentages of dispersed phase are calculated by considering that the co-surfactant belongs to the dispersed phase.

The aqueous phase includes from 0.01 to 50% by weight, preferably from 1 to 30% by weight and most particularly from 4 to 20% by weight of co-surfactant.

According to an embodiment, the dispersed phase of the nanoemulsion is grafted at the surface with molecules of interest such as biological ligands, in order to increase the specific targeting towards steroidal organs, or for targeting another organ. Such grafting allows specific recognition of certain cells or of certain organs. Preferably, the surface grafting is carried out by coupling molecules of interest or their precursors with an amphiphilic compound, notably with the co-surfactant. The nanoemulsion then comprises a grafted co-surfactant. In this case, the co-surfactant plays the role of a spacer allowing accommodation of the molecules of interest at the surface.

The molecules of interest may for example be:
Biological targeted ligands such as antibodies, peptides, saccharides, aptamers, oligonucleotides or compounds such as folic acid;
A furtivity agent: an entity added so as to give the nanoemulsion furtivity towards the immune system, to increase its circulation time in the organism, and to slow down its elimination.

For example, when the biological ligand is a peptide comprising one or several cysteines, the grafting to the alkylene oxide chain of the surfactant may be ensured by thiol-maleimide coupling.

Imaging Agent

In an embodiment, the formulation used comprises an imaging agent, which advantageously allows viewing of the distribution of the droplets in the body of the patient and therefore the distribution of the hormone and/or of the hormone agonist or antagonist.

The imaging agent may notably be used in imagings of the type:
positron emission tomography (PET) (the imaging agent may be a compound comprising a radionuclide, such as ¹⁸F, ¹¹C, a chelate of metal cations ⁶⁸Ga, ⁶⁴Cu),
single photon emission computed tomography (SPECT) (the imaging agent may be a compound comprising a radionuclide for example ¹²³I, or a chelate of ^{99m}Tc or ¹¹¹In),
magnetic resonance imaging (MRI) (the imaging agent may be a chelate of gadolinium or a magnetic nanocrystal such as a nanocrystal of iron oxide, manganese oxide or of iron-platinum FePt),
optical imaging or X-ray imaging (the imaging agent may be a lipophilic fluorophore or a contrast agent, for example an iodinated molecule such as iopamidol, amidotrizoate, or gold nanoparticles).

Preferably, the imaging agent is a lipophilic fluorophore giving the possibility of producing optical imaging.

The nature of the lipophilic fluorophore(s) which may be used, is not critical from the moment that they are compatible with imaging in vivo (i.e. they are biocompatible and non-toxic). Preferably, the fluorophores used as an imaging agent, absorb and emit in visible light and in the near infrared, in particular in the near infrared. Indeed, in order that the excitation light and the light emitted by the fluorophore may better cross the tissues, fluorophores absorbing and emitting in the infrared, i.e. at a wavelength comprised between 640 and 900 nm should be used.

As a lipophilic fluorophore, mention may for example be made of the compounds described in chapter 13 (“Probes for Lipids and Membranes”) of the InVitrogen catalogue. More specifically, as a fluorophore, mention may notably be made of indocyanine green (ICG), analogs of fatty acids and functionalized phospholipids with a fluorescent group such as the fluorescent products sold under the trade names Bodipy® such as Bodipy® 665/676 (Ex/Em.); amphiphlic derivatives of dialkylcarbocyanines such as l,r-dioctadecyl-3,3,3′,3′-tetramethylindodicarbocyanine (DiD) perchlorate, for example sold under reference D-307; fluorescent probes derived from sphingolipids, steroids or lipopolysaccharides such as the products sold under the trade names BODIPY® TR ceramides, BODIPY® FL C5-lactosylceramide, BODIPY® FL C5-ganglioside, BODIPY® FL cerebrosides; amphiphilic derivatives of cyanines, rhodamines, fluoresceins or coumarins such as octadecyl rhodamine B, octadecyl fluorescein ester and 4-heptadecyl-7-hydroxycoumarin; and diphenylhexatriene (DPH) and its derivatives; the whole of these products being sold by Invitrogen.

According to a preferred embodiment of the invention, the fluorophore is indocyanine green or l,r-dioctadecyl-3,3,3′,3′-tetramethylindodicarbocyanine perchlorate.

Aqueous Phase

The aqueous phase of the nanoemulsion used in the invention preferably consists of water and/or of a buffer such as a phosphate buffer like for example PBS (Phosphate Buffer Saline) or of a saline solution, notably of sodium chloride.

According to an embodiment, the continuous aqueous phase also includes a thickening agent such as glycerol, saccharide, oligosaccharide or polysaccharide, a gum or further a protein, for example glycerol. Indeed, the use of a continuous phase with higher viscosity promotes emulsification and consequently allows reduction in the sonication time.

The aqueous phase advantageously includes from 0 to 50% by weight, preferably from 1 to 30% by weight and most particularly from 5 to 20% by weight of thickening agent.

Of course, the aqueous phase may further contain other additives, such as coloring agents, stabilizers and preservatives in a suitable amount.

The proportion of dispersed phase and of aqueous phase is highly variable. However, most often, the nanoemulsions will be prepared with 1 to 50%, preferably 5 to 40% and most particularly 10 to 30% by weight of a dispersed phase (i.e. the (optional oil/solubilizing lipid/amphiphilic lipid/co-surfactant/optional imaging agent) assembly) and with 50 to 99%, preferably 60 to 95% and most particularly 70 to 90% by weight of aqueous phase.

Nanoemulsion as a Gel

In an embodiment, the dispersed phase of the formulation used represents between 35 and 65% by weight, preferably between 45 and 64% by weight based on the total weight of the nanoemulsion.

In this embodiment, the nanoemulsion used appears as a <<gel>>. By the term of <<gel>> is usually meant a thermodynamically stable solid-liquid biphasic system, consisting of a dual three-dimensional continuous interpenetrated network, one being solid and the second one liquid. Such a gel is a liquid-solid biphasic system for which the solid network retains a liquid phase. Although gels may be considered as solids, they have properties specific to solids (structural rigidity, elasticity upon deformation) and specific to liquids (vapor pressure, compressibility and electrical conductivity).

In the case of a nanoemulsion as a gel, the three-dimensional network is formed by the droplets, the interstices between droplets being filled with continuous phase. The bonds between the units of the network, i.e. the droplets, are generally based on non-covalent interactions of the hydrogen bond type, Van der Waals interactions, or further electrostatic interactions (ion pairs). These interactions mainly exist between the co-surfactants of adjacent droplets.

A nanoemulsion as a gel therefore exhibits resistance to pressure and is capable of keeping a definite shape, which may be advantageous depending on the administration form and/or route.

In order to demonstrate that the nanoemulsion is in the form of a gel, rheological studies may be conducted, allowing evaluation of the viscoelastic properties, and/or more structural studies showing the bonds between the droplets forming the three-dimensional network (X-ray, neutron diffraction, . . . ). Indeed, a nanoemulsion as a gel has viscosity and a larger elasticity coefficient than a liquid nanoemulsion. The nanoemulsion as a gel may, depending on the droplet concentration and therefore on the mass fraction in the dispersed phase, be found in the state of a viscous liquid, a viscoelastic solid or an elastic solid. As compared with the aqueous dispersant phase, for which the viscosity is close to that of water (1 mPa·s at 25° C.), the nanoemulsion is considered as a viscous liquid when its viscosity is ten times higher than that of water, i.e. >10 mPa·s at 25° C. Moreover, when it is proceeded with rheological measurement of the G′ (shearing conservation modulus) and G″ (*shearing loss modulus) moduli, it is considered that the nanoemulsion is in the form of a viscous liquid when G″>G′. When G′ becomes close to G″, the nanoemulsion is in the state of a viscoelastic solid. When G″<G′, it is in the state of an elastic solid. In this embodiment, the nanoemulsion preferably appears in the viscous liquid or viscoelastic solid state, since viscosity is sufficiently moderate in these states for allowing applications involving administration by injection. The viscosity and the elasticity coefficient may be measured by a cone-plate rheometer or by a Couette rheometer. The viscosity of a liquid nanoemulsion is generally less than 1 poise, or even often less than 0.01 poises. The nanoemulsion used in this embodiment of the invention generally has a viscosity of more than 1 poise, and may have a viscosity ranging up to that of a solid (more than 1,000 poises). The nanoemulsion of the present invention generally has a viscosity from 1 to 1,000 poises, preferentially from 1 to 500 poises and even more preferentially between 1 and 200, these values being given at 25° C. A viscosity of more than 1 poise is actually suitable for having the droplets of the dispersed phase form a three-dimensional network inside the continuous phase. Indeed, it was seen that below 1 poise, the droplets are generally not sufficiently close to each other. Beyond 1,000 poises, a quasi-solid system is obtained. The nanoemulsion is then too viscous which makes its use difficult. Also, when the elasticity coefficient is generally less than 10 in the case of a liquid nanoemulsion, the elasticity coefficient of a nanoemulsion as a gel is generally less than 10. Structural studies, notably X-ray or neutron diffractions, also allow differentiation of the organization of a liquid nanoemulsion from the organization of a nanoemulsion as a gel. Indeed, the peaks of the diffractrogram obtained for a liquid nanoemulsion are characteristic of the structure of the dispersed phase droplets (large diffraction angles characteristic of short distances), while the peaks of the diffractogram of a nanoemulsion as a gel are characteristic not only of the structure of the droplets (large diffraction angles characteristic of short distances) but also of the organization of these droplets in a three-dimensional network (low diffraction angles characteristic of larger distances).

The nanoemulsion used in this embodiment of the invention is advantageously in the form of a dispersible gel, i.e. the droplets forming the three-dimensional network may be released in the continuous phase under certain conditions by <<degelling>> of the gel system, also designated as <<disaggregation>> in the present application. Disaggregation is observed by adding a continuous phase to the gel, by contact with physiological fluids during administration of the nanoemulsion or by an increase in the temperature. Indeed, adding the continuous phase causes a difference in osmotic pressure between the inside of the gel and the continuous phase. The system will therefore tend to reduce, as far as cancel, this osmotic pressure difference by releasing the droplets in the excess of continuous phase, until a homogeneous droplet concentration is obtained in the whole of the volume of continuous phase. Also, sufficiently increasing the temperature of the system amounts to giving the different droplets a thermal energy greater than the energies set into play in the bonds, for example the hydrogen bonds, and thus to breaking these bonds and releasing the droplets of the three-dimensional network. These temperatures depend on the composition of the gel and more particularly on the size of the droplets and on the length of the polyalkoxylated chains of the co-surfactant. Disaggregation of the nanoemulsion as a gel may be tracked by X-ray diffraction, by differential scanning calorimetry (DSC) or by nuclear magnetic resonance (NMR). By tracking with X-ray diffraction, the disaggregation of the nanoemulsion as a gel, a time-dependent change in the spectrogram is observed, i.e. a reduction in the intensity of small angles (characteristic of the organization of the droplets as a three-dimensional network) (as described in Matija Tomsic, Florian Prossnigg, Otto Glatter ‘Journal of Colloid and Interface Science’ Volume 322, Issue 1, 1 Jun. 2008, Pages 41-50). The disaggregation may also be tracked with DSC. A peak appears on the thermogram during the (nanoemulsion as a gel)/(liquid nanoemulsion) transition during the rise in temperature. Finally, an NMR may also allow tracking of the disaggregation by measuring the diffusion coefficient associated with each droplet by distinguishing a liquid nanoemulsion from a nanoemulsion as a gel. Indeed, the diffusion coefficient is highly significantly reduced in the case of a nanoemulsion as a gel (it is then generally less than 0.01 μm²/s), where the system is set. (WESTRIN B. A.; AXELSSON A.; ZACCHI G. ‘Diffusion measurement in gels’, Journal of Controlled Release 1994, Vol. 30, no. 3, pp. 189-199).

Emulsion in which the Droplets are Bound Together Covalently

In an embodiment, the formulation used comprises a surfactant of the following formula (I):

(L₁-X₁—H₁—Y₁)ᵥ-G-(Y₂—H₂—X₂-L₂)_{w} (I),

wherein:
L₁ and L₂ represent independently lipophilic groups,
X₁, X₂, Y₁, Y₂ and G represent independently a binding group,
H₁ and H₂ represent independently hydrophilic groups comprising a polyalkoxylated chain,
v and w are independently an integer from 1 to 8,
   and the droplets of the dispersed phase are bound together covalently with the surfactant of formula (I).

The surfactant of formula (I) is partly located in the continuous aqueous phase and partly in the dispersed phase. Indeed, the surfactant of formula (I) comprises two lipophilic groups (L₁ and L₂) and two hydrophilic groups (H₁ and H₂). The hydrophilic groups are in majority located at the surface of the droplets, in the continuous aqueous phase while the lipophilic groups are located in the droplets of the formulation.

More specifically, the lipophilic group L₁ is located in certain droplets, and the group L₂ in adjacent droplets. The droplets are therefore bound covalently together by the —(X₁—H₁—Y₁)ᵥ-G-(Y₂—H₂—X₂)_{w}— group of the surfactant of formula (I). FIGS. 1 and 2 illustrate the positioning of the surfactant for formula (I) when v and w represent 1 and when v and w represent 2 respectively.

The groups X₁ and X₂ are binding groups binding the lipophilic and hydrophobic groups. The group G is a binding group between both [lipophilic-hydrophilic] portions of the surfactant of formula (I). The groups Y₁ and Y₂ are binding groups binding the group G to both of these [lipophilic-hydrophilic] portions.

The formulation used in this embodiment may advantageously be shaped (for example by placing it in a mould or a container having a given shape), and remain in the desired shape depending on the desired application. This embodiment of the invention is therefore particularly suitable when the formulation is used in the form of a gelatin capsule, of a gel, of a vaginal suppository or a patch.

On the other hand, it resists to dilution into an aqueous phase. More specifically, when an aqueous phase is added to this formulation, the latter retains its shape and is not diluted. In the medium, the formulation comprising droplets is observed on the one hand and an aqueous phase essentially free of droplets is observed on the other hand.

Without intending to be bound to a particular theory, it seems that these properties of this formulation may be explained by the presence of covalent bonds between the droplets, which provide very strong cohesion together.

In an embodiment, in the aforementioned formula (I):
L₁ and L₂ are independently selected from:
   a group R or R—(C═O)—, wherein R represents a linear hydrocarbon chain comprising from 11 to 23 carbon atoms,
   an ester or amide of fatty acids comprising from 12 to 34 carbon atoms and of phosphatidylethanolamine, such as distearyl phosphatidylethanolamine (DSPE), and
   a poly(propylene oxide), and/or
X₁, X₂, Y₁ and Y₂ are independently selected from:
   a simple bond,
   a group Z selected from —O—, —NH—, —O(OC)—, —(CO)O—, —(CO)NH—, —NH(CO)—, —O—(CO)—O—, —NH—(CO)—O—, —O—(CO)—NH— and —NH—(CO)—NH,
   a group Alk being an alkylene comprising from 1 to 6 carbon atoms, and
   a group Z-Alk, Alk-Z, Alk-Z-Alk or Z-Alk-Z wherein Alk and Z are as defined above and wherein both groups Z of the Z-Alk-Z group are identical or different,
and/or
H₁ and H₂ are independently selected from a poly(ethylene oxide) typically comprising from 3 to 500 ethylene oxide units, preferably from 20 to 200 ethylene oxide units, and/or
G comprises at least one group G′ having one of the following formulae (the groups Y₁ and Y₂ being bound on the left and on the right of the formulae described below:

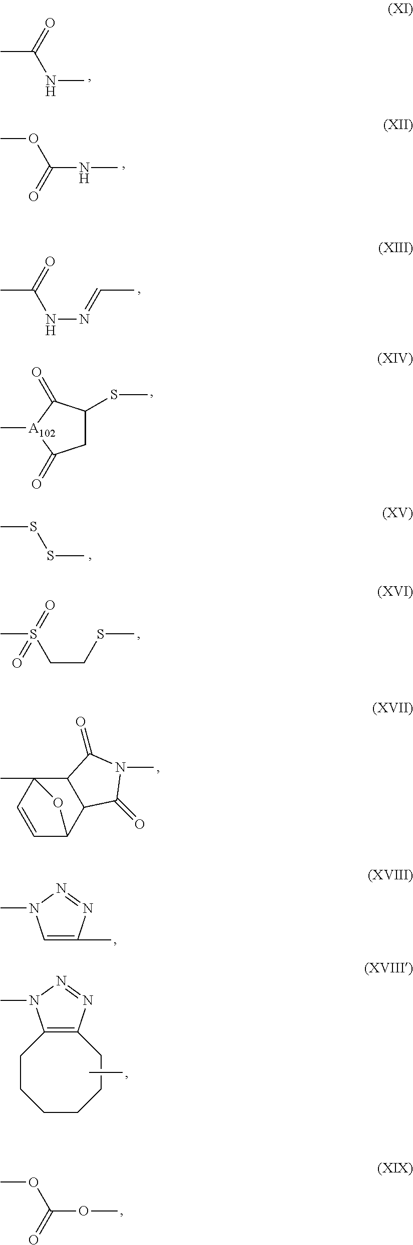

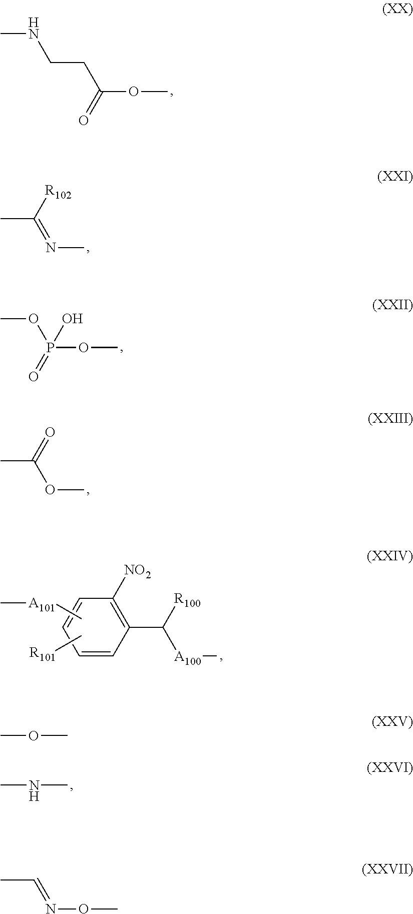

wherein A₁₀₂ represents CH or N, R₁₀₂ represents H or a linear hydrocarbon chain comprising from 1 to 6 carbon toms, A₁₀₁ represents —O—, —NH—(CO)— or —O—(CO)—, R₁₀₀ represents H or a methyl, A₁₀₀ represents —O— or —NH— and R₁₀₁ represents H, Me or —OMe. By the formula

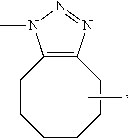

is meant that the group Y₂ may be connected to any of the six atoms of the cyclic octyl group and by the

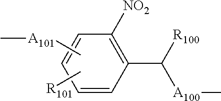

formula, is meant that the groups A₁₀₁ et R₁₀₁ may be connected to any of the four atoms of the phenyl group.

Notably, v and w represent independently 1 or 2. Preferably v and w represent 1.

The group G may comprise one or several of the G′ groups defined above.

Thus, in a first embodiment, the group G consists of a group G′. In this embodiment, in formula (I), v and w represent 1.

In a second embodiment, the group G fits the formula -G′-Y₃-G′- wherein:
Y₃ represents a binding group, notably selected from:
   a simple bond,
   a group Z selected from —O—, —NH—, —O(OC)—, —(CO)O—, —(CO)NH—, —NH(CO)—, —O—(CO)—O—, —NH—(CO)—O—, —O—(CO)—NH— and —NH—(CO)—NH,
   an Alk group being an alkylene comprising from 1 to 6 carbon atoms, and
   a group Z-Alk, Alk-Z, Alk-Z-Alk or Z-Alk-Z wherein Alk and Z are as defined earlier and wherein both groups Z of the Z-Alk-Z group are identical or different,
each of the G′ groups represent independently an aforementioned group of formula (XI) to (XXVI), and preferably both groups G′ of formula -G′-Y₃-G′- are identical.

In this embodiment, in formula (I), v and w represent 1.

This embodiment is particular of interest when both groups G′ are identical and comprise a cleavable function. Indeed, it is then sufficient to cleave only one of the two functions for breaking the covalent bonds between the droplets of the formulation.

In a third embodiment, the group G is a dendrimer comprising (v+w) G′ groups. The group G may notably be a dendrimer comprising several G′ groups, such as a dendrimer comprising a polyamidoamine (PAMAM) group. For example, the group G may have one of the following formulae (XXX) to (XXXIII), which comprise:
4 G′ groups with formula (XXVI). v and w represent 2.
4 G′ groups with formula (XXIV) wherein R₁₀₁ represents —O-Me, A₁₀₁ represents —NH—, R₁₀₀ represents a methyl and A₁₀₀ represents —NH—. v and w represent 2.
4 groups G′ of formula (XIV). v and w represent 2.
16 G′ groups with formula (XXVI). v and w represent 8.

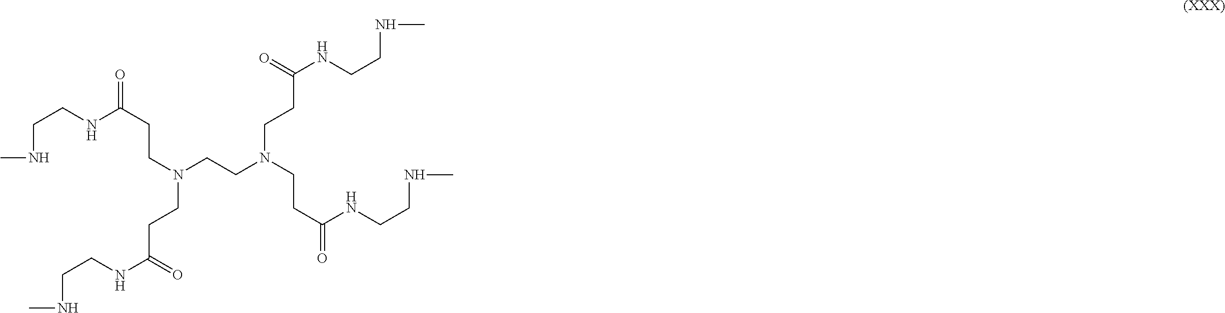

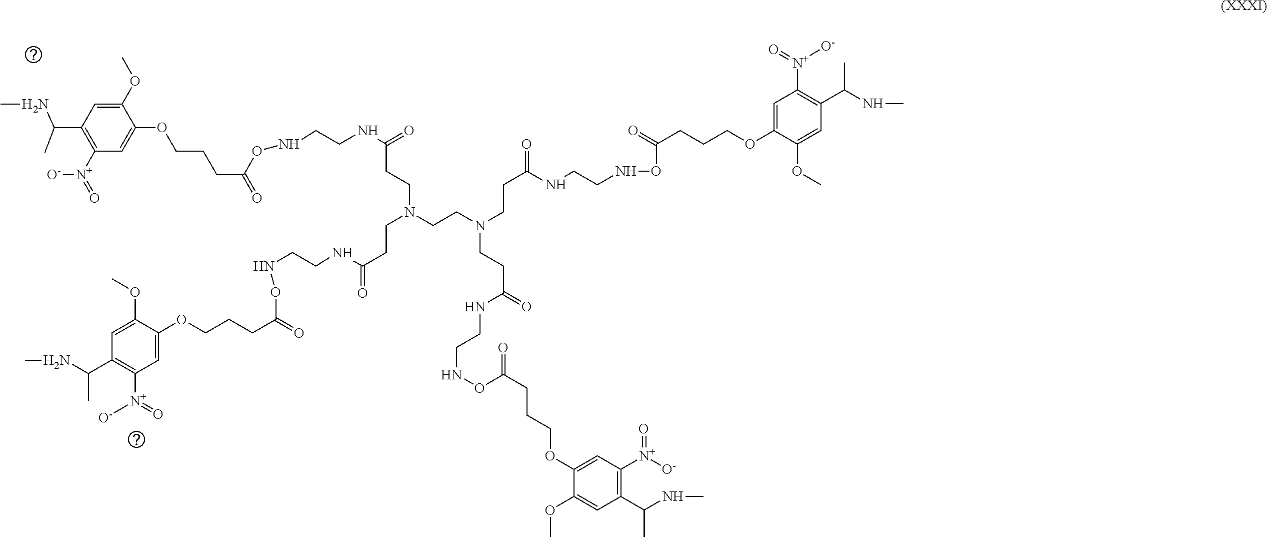

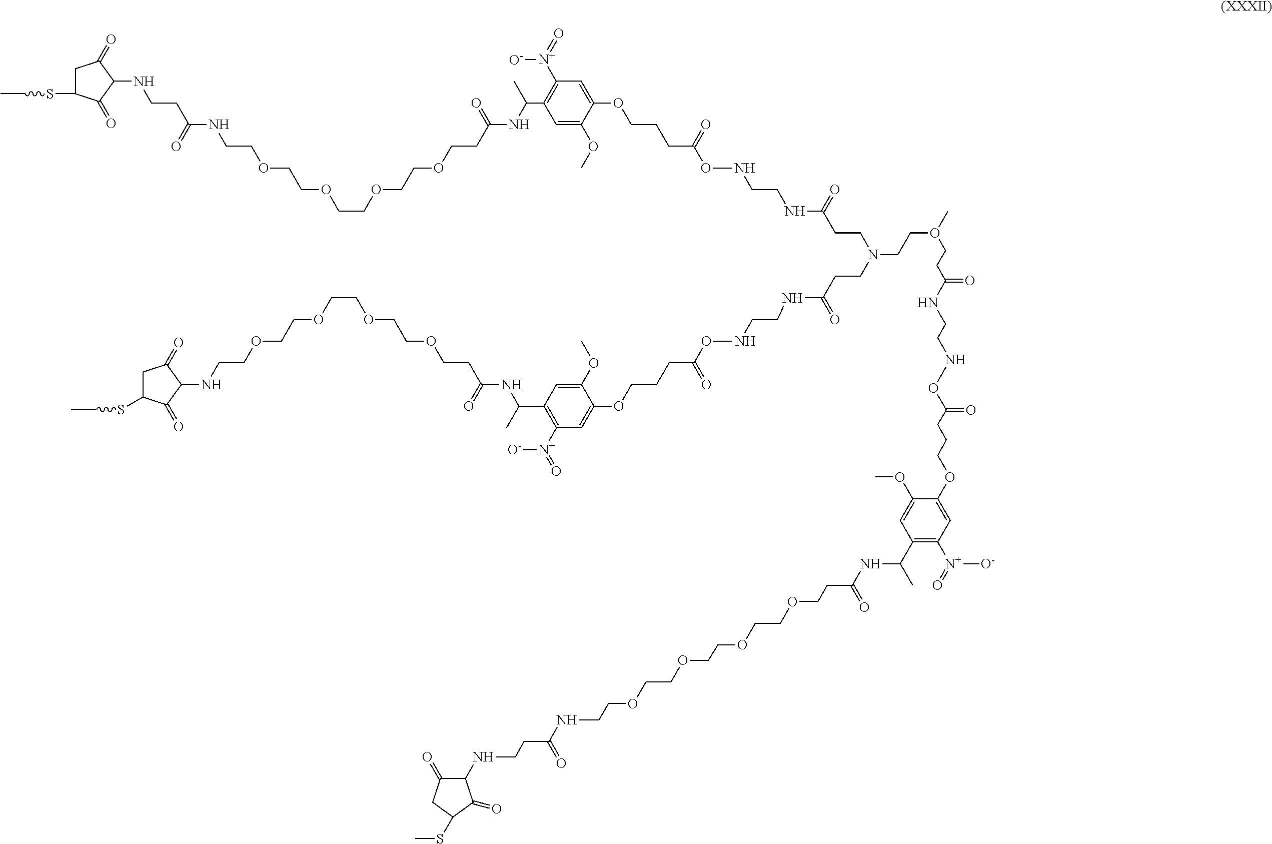

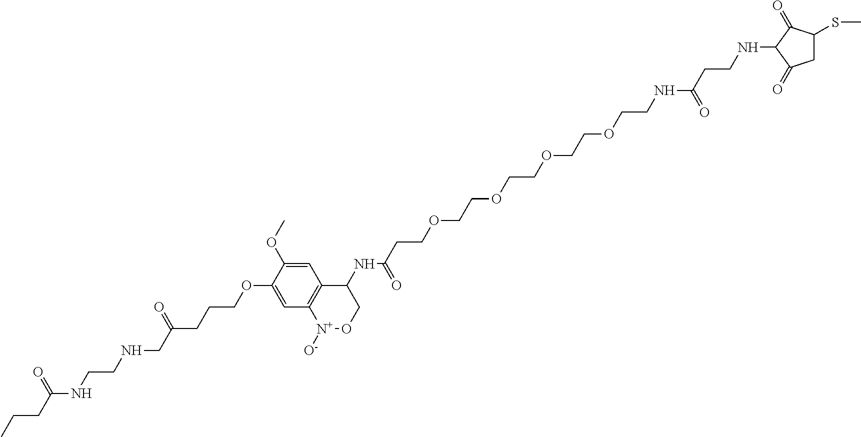 [SMILES: CCCC(=O)NCCNCC(=O)CCCOc1cc2c(cc1OC)C(NC(=O)CCOCCOCCOCCOCCNC(=O)CCNC1C(=O)CC(SC)C1=O)CO[NH+]2[O-]]

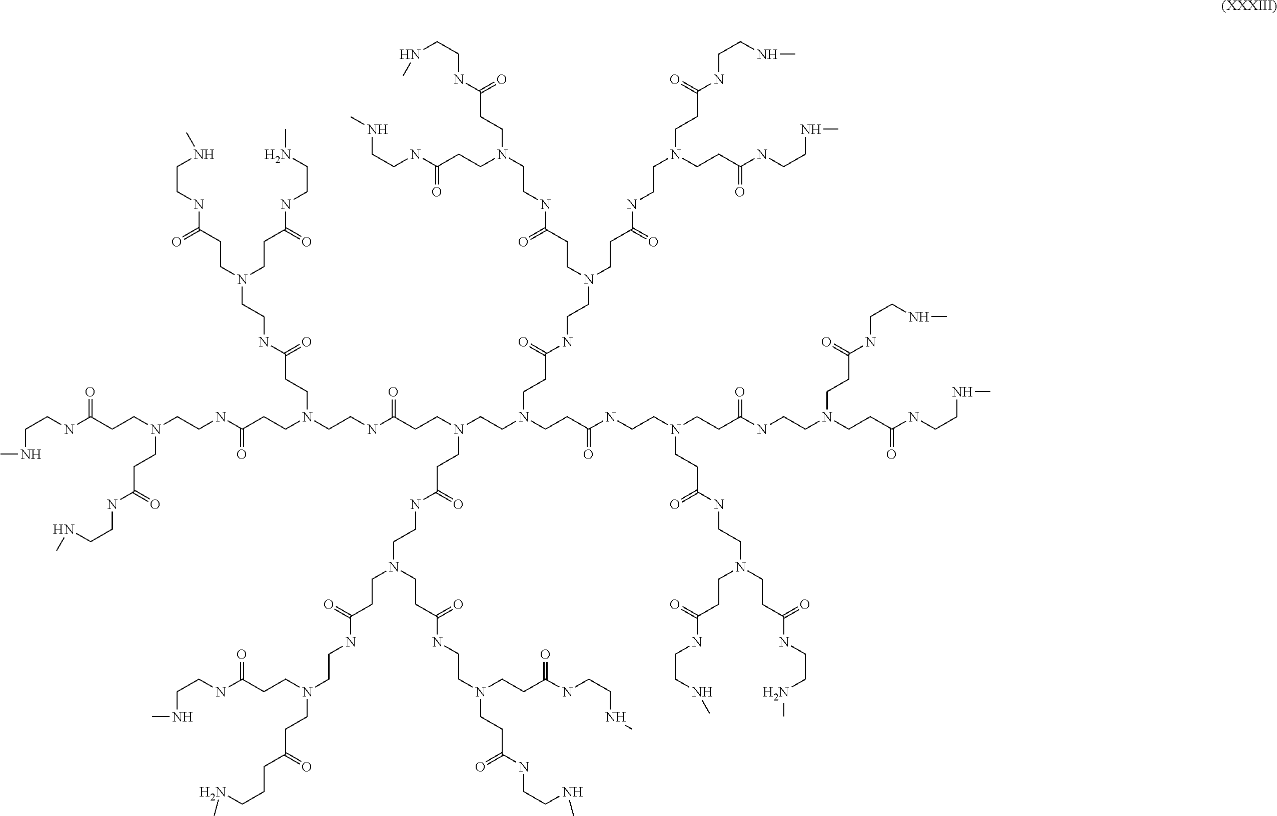

When L₁ and/or L₂ represent a group R—(C═O)—, wherein R represents a linear hydrocarbon chain comprising from 11 to 23 carbon atoms, L₁ and/or L₂ represent groups stemming from a fatty acid comprising from 12 to 24 carbon atoms.

By <<L₁ and L₂ represent an ester or an amide of fatty acids comprising from 12 to 24 carbon atoms and of phosphatidylethanolamine>>, is meant that they represent a group of formula:

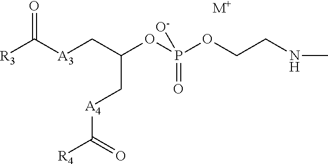

wherein
R₃ and R₄ represent independently a linear hydrocarbon chain comprising from 11 to 23 carbon atoms,
A₃ and A₄ represent O or NH, and
M represents H or a cation.

Preferably, L₁ and L₂ are identical and/or X₁ and X₂ are identical and/or H₁ and H₂ are identical. Particularly preferred surfactants of formula (I) are those in which L₁ and L₂ are identical, X₁ and X₂ are identical and H₁ and H₂ are identical. These surfactants are actually symmetrical compounds and are therefore generally easier to synthesize, and therefore less expensive.

In an embodiment, in the aforementioned formula (I), the radicals L₁-X₁—H₁— and/or L₂-X₂—H₂— consist in one of the groups of the following formulae (the group Y₁ or Y₂ being bound on the right of the formulae described below):

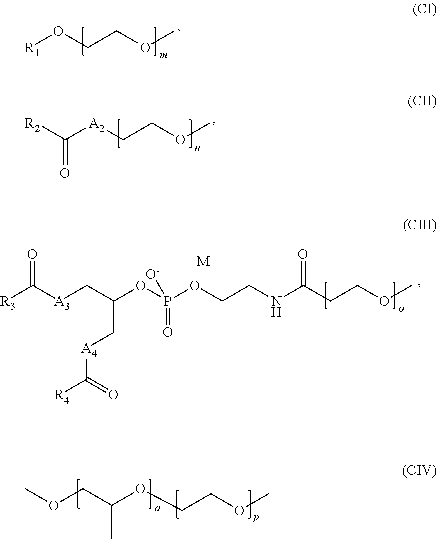

wherein:
R₁, R₂, R₃ and R₄ represent independently a linear hydrocarbon chain comprising from 11 to 23 carbon atoms.
A₁, A₂, A₃ and A₄ represent O or NH,
m, n, o and p represent independently integers from 2 to 500, preferably 20 to 200, and
a represents an integer from 20 to 120,
M represents H or a cation.

The radicals L₁-X₁—H₁— and/or L₂-X₂—H₂— of formula (CII) are preferred. Indeed, they are easy to prepare (notably by forming an ester or an amide between a fatty acid and a poly(ethylene glycol) derivative. Further, a formulation comprising a surfactant comprising a radical L₁-X₁—H₁— and/or L₂-X₂—H₂— of formula (CII) may generally be prepared with a larger amount of this surfactant than a formulation comprising a surfactant comprising a radical L₁-X₁—H₁— and/or L₂-X₂—H₂— of formula (CIII). Now, the higher the proportion of surfactant of formula (I) in the formulation, the larger is the cohesion between the droplets, and the more the formulation retains its shape and resists dilution. Thus, both of these properties may further be exacerbated for a formulation comprising a surfactant comprising a radical L₁-X₁—H₁— and/or L₂-X₂—H₂— of formula (CII).

The radicals L₁-X₁—H₁— and/or L₂-X₂—H₂— of formula (CII) with A₂ representing NH, are particularly preferred, since the surfactants comprising such radicals give the possibility of avoiding the leak of optionally present lipophilic agents of interest, outside the droplets of the formulation more efficiently than surfactants comprising L₁-X₁—H₁— and/or L₂-X₂—H₂— radicals of formula (CII) with A₂ representing O.

In an embodiment, in formula (I), v and w represent 1, L₁ and L₂ are independently R—(C═O)—, wherein R represents a linear hydrocarbon chain comprising from 11 to 23 carbon atoms, H₁ and H₂ are independently poly(ethylene oxides) comprising from 3 to 500 ethylene oxide units, X₁ and X₂ represent —O or —NH—, G consists of a group G′ representing —S—S— (the group of formula (XV) above) and Y₁ and Y₂ represent —CH₂—CH₂—NH—CO—CH₂—CH₂— (the group Alk-Z-Alk above with Alk representing —CH₂—CH₂— and Z representing —NH—(CO)—) and the surfactant of the formulation then has the following formula (I′):

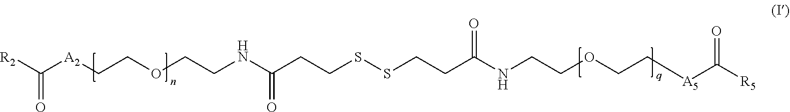

wherein:
R₂ and R₅ represent independently a linear hydrocarbon chain comprising from 11 to 23 carbon atoms, preferably 17 carbon atoms,
A₂ and A₅ represent O or NH, preferably NH, and
n and q represent independently integers from 3 to 500, preferably from 20 to 200.

In an embodiment, the groups H₁ and H₂ are independently selected from a poly(ethylene oxide) comprising more than 3 poly(ethylene oxide) units or even more than 20 units, notably more than 50 (in the aforementioned formulae, m, n, o, p and/or q are preferably greater than 3, or even than 20, notably more than 50).

In an embodiment, the group G of the surfactant of formula (I) of the formulation used comprises a function, notably at certain pHs (basic or acid pHs), cleavable by enzymes, by light (visible light, ultraviolet or infrared light) and/or beyond certain temperatures. Generally the group G then comprises a group G′ comprising a cleavable function.

For example:
the β-ketoaminoester function of the group G of formula (XX) is cleavable at an acid pH (typically around 5),
the disulfide function of the group G of formula (XV) is cleavable with ultraviolet light or with enzymes such as thioreductases,
the amide function of the group G of formula (XI) is cleavable by enzymes such as proteases,
the phosphate function of the group G of formula (XXII) is cleavable by enzymes such as phosphatases,
the imine function of the groups G of formula (XXI) and (XIII) are cleavable at an acid pH or beyond certain temperatures,
the cyclohexene ring of the group G of formula (XVII) is cleavable beyond certain temperatures (by retro Diels-Alder),
the carbonate function of the group G of formula (XIX) and the carbamate function of the group G of formula (XII) are cleavable at an acid pH.

One skilled in the art, considering his/her general knowledge, is aware of the functions which are cleavable and under which conditions. He/she is notably capable of selecting the function of the group G′ of the surfactant of formula (I) so that it is cleavable under the conditions encountered during administration of the formulation according to the invention.

Preferably, the ratio of the mass of surfactant of formula (I) over the mass of the whole (surfactant of formula (2)/co-surfactant) is greater than or equal to 15%. Indeed it has been observed that such formulations are easier to prepare.

Therapeutic Application

When it is administered, the formulation used in the invention preferentially targets steroidal organs (ovaries and adrenal glands).

Without intending to be bound to particular theories, this preferential targeting may be explained:
by the fact that the chemical compounds from the metabolism of the formulation used in the invention are useful as initial products for synthesizing hormones, and/or
by the fact that the formulation used in the invention is a synthetic mimic of lipoproteins, which convey the cholesterol to the organs in need of it, such as steroidal organs, such as for example VLDLs (very low density lipoproteins) or chylomycrons. Indeed, the droplets of the formulation according to the invention have:
a size, and
a composition (they comprise a glyceride derivative (solubilizing lipid) and optionally a phospholipid (amphiphilic lipid) which is again found in the aforementioned lipoproteins)
   similar to those of these lipoproteins. Once administered, the droplets of the formulation according to the invention will therefore be conveyed towards steroidal organs, like lipoproteins.

As explained above, the formulation used in the invention gives the possibility of limiting the doses of the hormone, of the hormone agonist or antagonist or of the mixture thereof, which are administered and allows limitation of the secondary effects due to their administration.

Moreover, by modulating the natures and concentrations of the amphiphilic and solubilizing lipids, of the co-surfactant, of the surfactant of formula (I) and of the oil and the size of the droplets of the dispersed phase, it is possible to control the kinetics for delivering the hormone, the hormone agonist or antagonist, notably in order to avoid concentration peaks.

The formulation may be used as such, or adapted to the targeted application, for example by dilution, for administration of the hormone, of the hormone agonist or antagonist or of the mixture thereof, in humans or in animals.

Because it may be exclusively prepared from approved constituents for humans, it is of interest for administration via a parenteral route. However, it is also possible to contemplate administration via other routes, notably via an oral route, a topical route, or a vaginal route. The oral and topical routes (in particular when the formulation used is in the form of a patch) are particularly preferred.

The formulation used may be in the form of a gelatin capsule, of a vaginal suppository, of a gel, of an emulsion, of a cream, of a patch, of a spray . . .

Typically, when it is administered via:
an oral route, it is in the form of a gelatin capsule or a capsule,
a buccal or sublingual route, it is in the form of a gelatin capsule, of a capsule, of a gel, of an emulsion,
a transdermal route, it is in the form of a gel, an emulsion, a patch, a dermal spray, an intranasal spray,
an intra-uterine route, it is in the form of a gel, a cream, a vaginal suppository,
an intramuscular route, it is in the form of a gel or of an emulsion.

The group of patients to which the formulation is administered is varied. Of course, for using the formulation for contraception, reproduction assistance, post-menopausal hormonal treatment and treatment of infertility due to adrenal dysregulation, the patients are women. Generally, for using the formulation for treating acne, the patients are adolescents and/or young adults (typically between 12 and 25 years old).

A hormonal treatment method comprising administration in mammals, preferably humans, which are in need of it with an efficient amount of the formulation as defined above, is also one of the objects of the present invention.

Preparation of the Nanoemulsion Used

Advantageously, the agents are incorporated into the emulsion as a solution, and the solvent is then separated, for example by evaporation. The solution contains the hormone, the hormone agonist or antagonist or the mixture thereof in a variable amount which may range up to its solubility limit. The selection of the solvant depends on the solubility of each hormone/agonist/antagonist. The solvents used may for example be methanol, ethanol, chloroform, dichloromethane, hexane, cyclohexane, DMSO, DMF or further toluene. Preferably, these are volatile solvents, preferably non-toxic for humans, preferably ethanol.

The nanoemulsion used may easily be prepared by dispersing suitable amounts of oily phase and of aqueous phase under the effect of shearing.

Typically, the different oily constituents, the hormone, the hormone agonist or antagonist or the mixture thereof are mixed first in order to prepare an oily premix for the dispersed phase of the emulsion. The preparation method typically comprises the following steps:
(i) preparing the oily phase comprising a solubilizing lipid, an amphiphilic lipid, the hormone, the hormone agonist or antagonist or a mixture thereof;
(ii) preparing an aqueous phase comprising a polyalkoxylate co-surfactant;
(iii) dispersing the oily phase in the aqueous phase under the action of sufficient shearing in order to form a nanoemulsion; and
(iv) recovering the thereby formed nanoemulsion.

The mixing may optionally be facilitated by putting into solution one of the constituents or the complete mixture in a suitable organic solvent. The organic solvent is then evaporated, in order to obtain a homogeneous oily premix for the dispersed phase.

Moreover, it is preferred to produce the premix (step (i)) at a temperature at which the whole of the ingredients is liquid.

Advantageously, the oily phase is dispersed into the aqueous phase in the liquid state. If one of the phases solidifies at room temperature, it is preferable to achieve the mixture with one or preferably both phases heated to a temperature greater than or equal to the melting temperature.

The emulsification under the effect of shearing is preferably achieved by means of a sonicator or a microfluidizer. Preferably, the aqueous phase and then the oily phase are introduced in the desired proportions in a suitable cylindrical container and then the sonicator is immersed in the medium and started for a sufficient time in order to obtain a nanoemulsion, most often within a few minutes.

A homogeneous nanoemulsion is then obtained, in which the average diameter of the oil drops is generally greater than 10 nm and less than 200 nm, preferably between 30 and 150 nm.

Preferably, the zeta potential is less than 20 mV in absolute value, i.e. comprised between −20 mV and 20 mV.

Before conditioning, the emulsion may be diluted and/or sterilized, for example by filtering or by dialysis. This step gives the possibility of removing the possible aggregates which may have formed during the preparation of the emulsion.

The thereby obtained emulsion is ready-to-use, if necessary after dilution.

In the embodiments in which the nanoemulsion used comprises a co-surfactant grafted with a molecule of interest (for example a biological ligand), the preparation method typically comprises the following steps:
(i) preparing the oily phase comprising a solubilizing lipid, an amphiphilic lipid, the hormone, the hormone agonist or antagonist or the mixture thereof;
(ii) preparing an aqueous phase comprising a polyalkoxylated co-surfactant and a function which may react with the molecule of interest;
(iii) dispersing the oily phase in the aqueous phase under the action of sufficient shearing for forming a nanoemulsion;
(iv) adding to the nanoemulsion the molecule of interest under conditions allowing grafting between the function capable of reacting with the molecule of interest of the co-surfactant and the molecule of interest,
(v) recovering the thereby formed nanoemulsion.

Grafting is therefore typically carried out after forming the nanoemulsion, which may be recommended when the chemical reactions used are compatible with the colloidal stability of the emulsions, notably in terms of pH. Preferably the pH during the grafting reaction is comprised between 5 and 11.

In another embodiment, the preparation method comprises the following steps:
(i) providing a polyalkoxylated co-surfactant grafted with the molecule of interest,
(ii) preparing the oily phase comprising a solubilizing lipid, an amphiphilic lipid, the hormone, the hormone agonist or antagonist or a mixture thereof;
(iii) preparing an aqueous phase comprising the polyalkoxylated co-surfactant grafted with the molecule of interest;
(iv) dispersing the oily phase into the aqueous phase under the action of sufficient shearing for forming a nanoemulsion;
(v) recovering the thereby formed nanoemulsion.
   The polyalkoxylated co-surfactant grafted with the molecule of interest is generally obtained by putting the molecule of interest in contact with a co-surfactant comprising a function capable of reacting with the molecule of interest, under conditions allowing grafting between the functions capable of reacting with the molecule of interest of the co-surfactant and the molecule of interest.

In the embodiment in which the formulation used comprises a surfactant of formula (I), the latter may be prepared by a method comprising the putting into contact:
of an emulsion 1 comprising a continuous aqueous phase and a phase dispersed as droplets comprising an amphiphilic lipid and a surfactant of the following formula (LI):

L₁-X₁—H₁—Y₁-G₁ (LI),
with an emulsion 2 comprising a continuous aqueous phase and a phase dispersed as droplets comprising an amphiphilic lipid and a surfactant of the following formula (LII):

G₂-Y₂—H₂—X₂-L₂ (LII)
wherein L₁, X₁, H₁, Y₁, L₂, X₂, H₂ and Y₂ are as defined above, and G₁ and G₂ are groups which may react in order to form the group G as defined above,
   under conditions allowing reaction of the surfactants of formulae (LI) and (LII) in order to form the surfactants of formula (I) as defined above, whereby covalent bonds are formed between the droplets of the dispersed phase.

When the group G comprises a single group G′, the groups G₁ and G₂ are typically groups capable of reacting with each other in order to form the group G.

When the group G comprises several groups G′, the emulsions 1 and 2 are generally put into contact with a compound capable of reacting with the surfactants of formulae (LI) and (LII) in order to form the group G. This compound typically comprises at least v functions G′₁ which may react with the group G₁ and w functions G′₂ capable of reacting with the group G₂.

Thus, in the embodiment in which the group G fits the formula -G′-Y₃-G′- defined above, the method for preparing the formulation typically comprises the putting into contact:
of an emulsion 1 as defined above,
and of an emulsion 2 as defined above,
with a compound of formula G′₁-Y₃-G′₂ wherein Y₃ is as defined above, G′₁ is a group which may react with G₁ in order to form the first group G′ as defined above and G′₂ is a group which may react with G₂ in order to form the second group G′ as defined above (with a nature either identical or different from the first group G′),
   under conditions allowing reaction of the surfactants of formulae (LI) and (LII) and of the compound of formula G′₁-Y₃-G′₂ in order to form the surfactants of formula (I) in which the group G fits the formula -G′-Y₃-G′- defined above, whereby covalent bonds are formed between the droplets of the dispersed phase.

Also, in the embodiment defined above in which the group G is a dendrimer comprising (v+w) groups G′, the method for preparing the formulation typically comprises the putting into contact:
of an emulsion 1 as defined above,
of an emulsion 2 as defined above,
with a dendrimer of formula (G′₁)ᵥ-Y₄-(G′₂)_{w} in which v and w are as defined above, G′₁ is independently a group capable of reacting with G₁ in order to form a group G′ as defined above, and G′₂ is independently a group capable of reacting with G₂ in order to form a group G′ as defined above (each G′ being of a nature identical with or different from the other groups G′) and Y₄ is the backbone of a dendrimer,
   under conditions allowing the reaction of the surfactants of formulae (LI) and (LII) and of the dendrimer of formula (G′₁)ᵥ-Y₄-(G′₂)_{w} in order to form the surfactant of formula (I) in which the group G is a dendrimer comprising (v+w) groups G′, whereby covalent bonds are formed between the droplets of the dispersed phase.
   For example, in order to form a group G having the formulae (XXX), (XXXI), (XXXII) and (XXXIII), the compound of formula (G′₁)ᵥ-Y₄-(G′₂)_{w} may have one of the following formulae (XXX′), (XXXI′), (XXXII′) and (XXXIII') respectively:

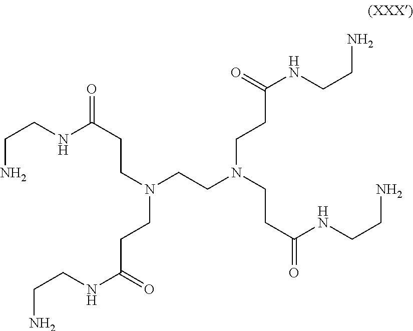 [SMILES: NCCNC(=O)CCN(CCC(=O)NCCN)CCN(CCC(=O)NCCN)CCC(=O)NCCN]

(wherein G′₁ and G′₂ represent NH₂ and v and w represent 2),

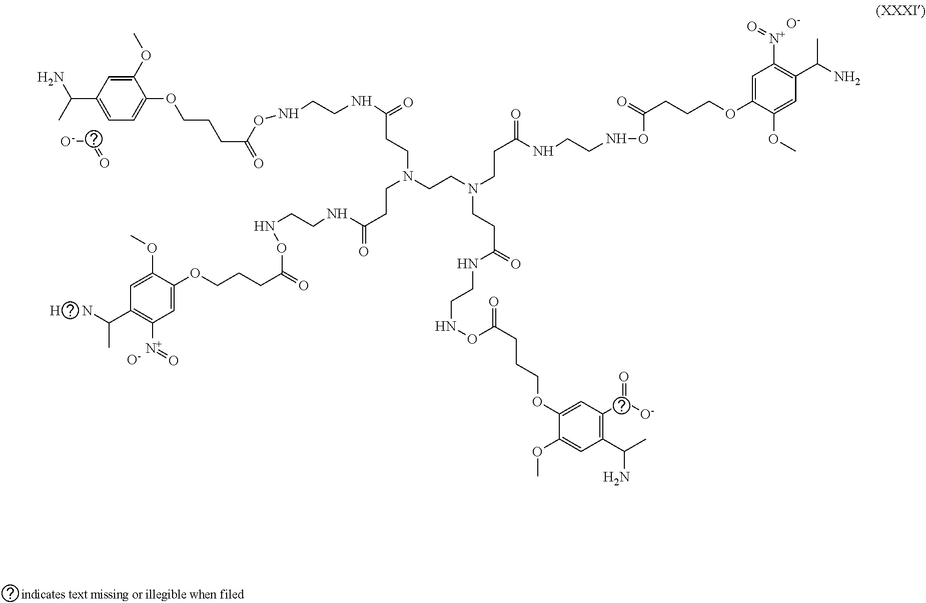

(wherein G′₁ and G′₂ represent NH₂ and v and w represent 2),

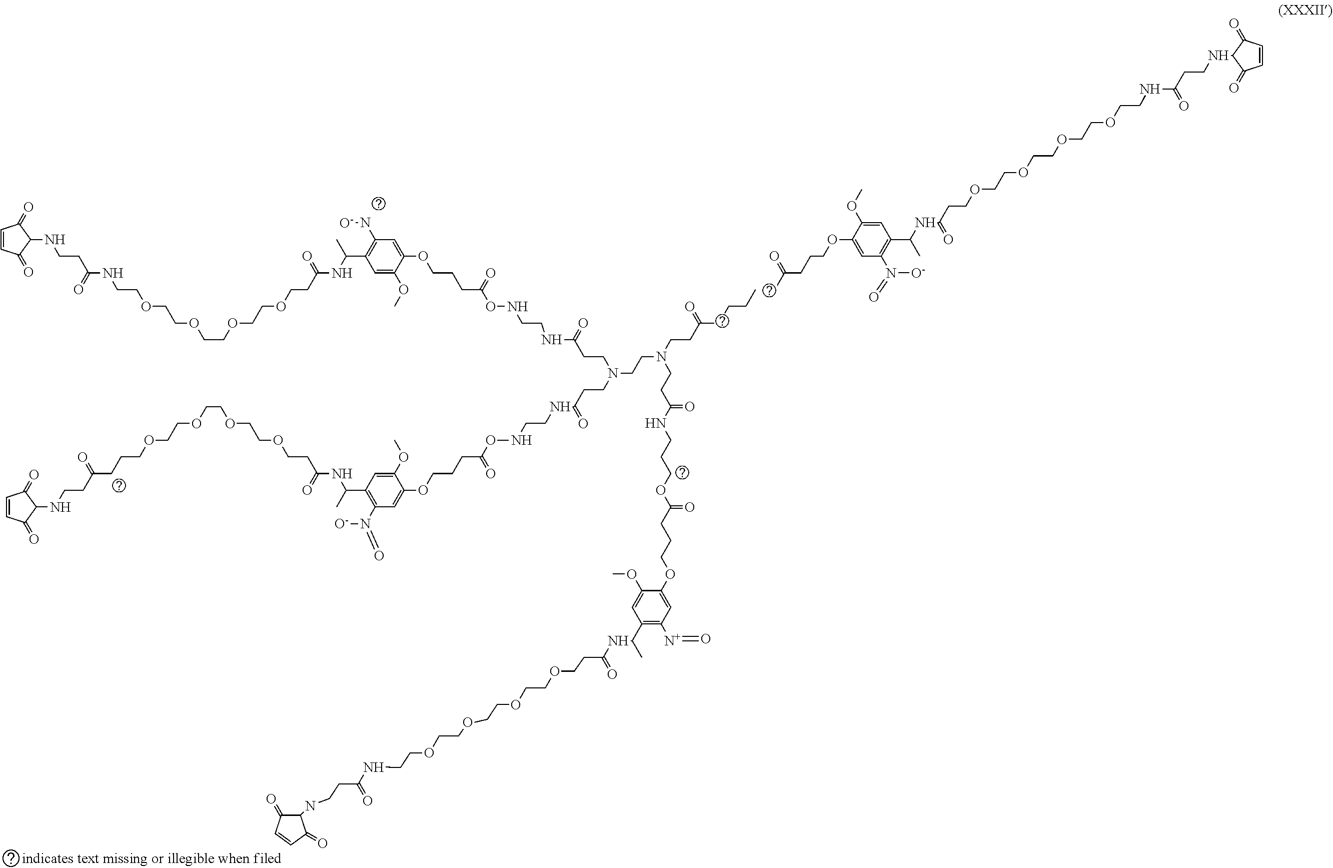

(wherein G′₁ and G′₂ represent

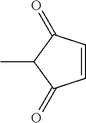

and v and w represent 2),

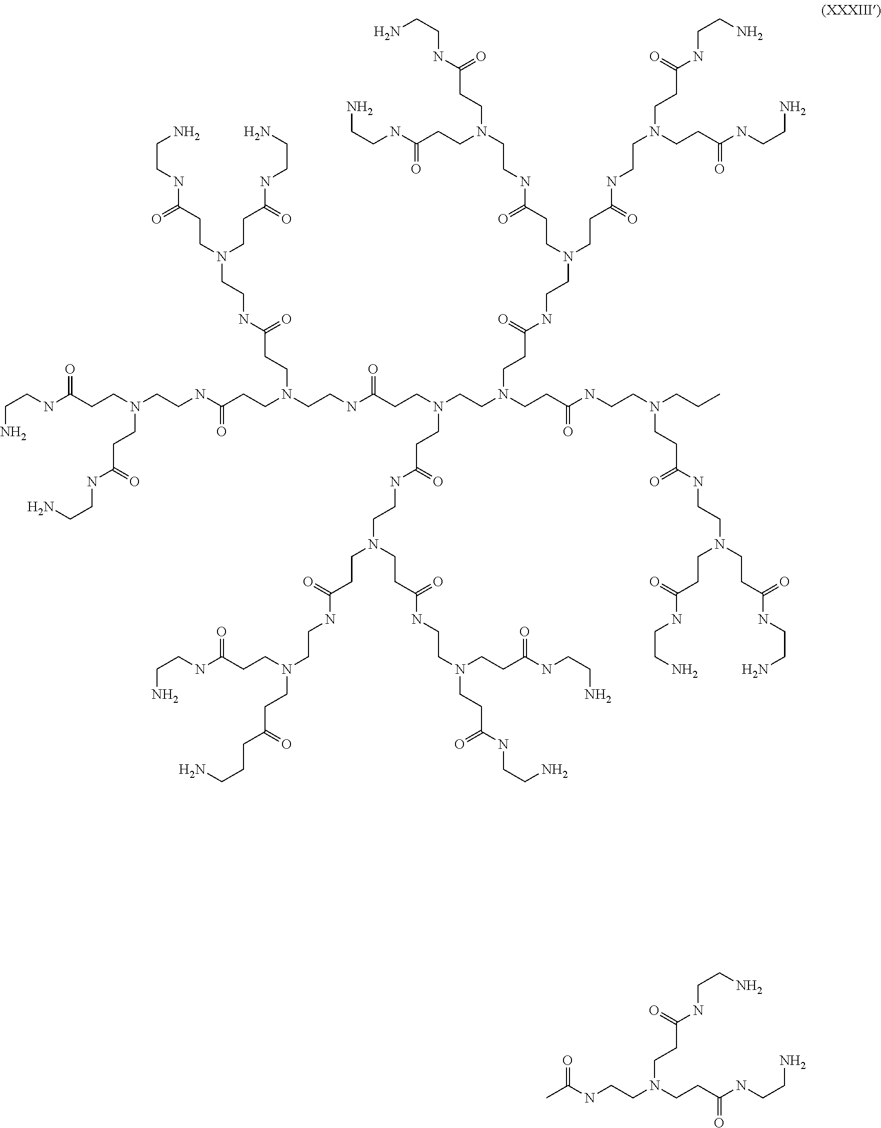 [SMILES: CCCN(CCNC(=O)CCN(CCC(=O)NCCN(CCC(=O)NCCN(CCC(=O)NCCN)CCC(=O)NCCN)CCC(=O)NCCN(CCC(=O)NCCN)CCC(=O)NCCN)CCN(CCC(=O)NCCN(CCC(=O)NCCN(CCC(=O)CCCN)CCC(=O)NCCN)CCC(=O)NCCN(CCC(=O)NCCN)CCC(=O)NCCN)CCC(=O)NCCN(CCC(=O)NCCN(CCC(=O)NCCN)CCC(=O)NCCN)CCC(=O)NCCN(CCC(=O)NCCN)CCC(=O)NCCN)CCC(=O)NCCN(CCC(=O)NCCN)CCC(=O)NCCN ; CC(=O)NCCN(CCC(=O)NCCN)CCC(=O)NCCN]

(wherein G′₁ and G′₂ represent NH₂ and v and w represent 8).

Considering his/her general knowledge in chemistry, one skilled in the art is capable of selecting the nature of the groups G′₁, G′₂, Y₃, Y₄, G₁ and G₂ to be used for forming the group G and the conditions allowing the reaction. The customary organic chemistry reactions may be followed, notably those described in <<Comprehensive Organic Transformations: A Guide to Functional Group Preparations>> of Richard C. Larock edited by John Wiley & Sons Inc., and the references which are quoted therein. Thus, the examples of groups G₁ and G₂ below are mentioned as an illustration and not as a limitation.

Typically, when the group G consists of a group G′, the groups G₁ and G₂ of the compounds of formula (LI) and (LII) may for example be selected as follows:
G₁ represents a thiol (—SH) and G₂ represents:
   either a maleimide, a surfactant of formula (I) in which G comprises a group G′ representing a group of formula (XIV) wherein A₁₀₂ represents N then being formed,
   or a vinylsulfone, a surfactant of formula (I) wherein G comprises a group G′ representing a group of formula (XVI) then being formed,
   or an —S—S-pyridinyl or —SH group, a surfactant of formula (I) in which G comprises a group G′ representing a group of formula (XV) then being formed,
G₁ represents a hydroxyl and G₂ represents —COOH or an activated ester, a surfactant of formula (I) wherein G comprises a group G′ representing a group of formula (XXIII) then being formed,
G₁ represents an amine —NH₂ and G₂ represents —COOH or an activated ester, a surfactant of formula (I) wherein G comprises a group G′ representing a group of formula (XI) then being formed,
G₁ represents a hydroxyl and G₂ represents an activated carbonate, a surfactant of formula (I) wherein G comprises a group G′ representing a group of formula (XIX) then being formed,
G₁ represents an amine —NH₂ and G₂ represents an activated carbonate, a surfactant of formula (I) wherein G comprises a group G′ representing a group of formula (XII) then being formed,
G₁ represents an amine —NH₂ and G₂ represents an aldehyde —CHO, a surfactant of formula (I) wherein G comprises a group G′ representing a group of formula (XXI) then being formed,
G₁ represents a hydrazide of formula —(C═O)—NH—NH₂ and G₂ represents a group —(C═O)—R10₂, a surfactant of formula (I) wherein G comprises a group G′ representing a group of formula (XIII) then being formed,
G₁ represents an alkyne and G₂ represents an azide, a surfactant of formula (I) wherein G comprises a group G′ representing a group of formula (XVIII) then being formed,
G₁ represents a cyclo-octyne and G₂ represents an azide, a surfactant of formula (I) wherein G comprises a group G′ representing a group of formula (XVIII′) then being formed,
G₁ represents a furane and G₂ represents a maleimide, a surfactant of formula (I) wherein G comprises a group G′ representing a group of formula (XVII) then being formed,
G₁ represents an aldehyde and G₂ represents an amine, a surfactant of formula (I) wherein G comprises a group G′ representing a group of formula (XXI) then being formed,

G₁ represents a phosphate of formula —O—P(═O)(OH)₂ and G₂ represents a hydroxyl, a surfactant of formula (I) wherein G comprises a group G′ representing a group of formula (XXIII) then being formed,
G₁ represents a good leaving group LG and G₂ represents a group of the following

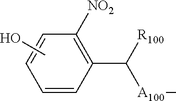

formula, a surfactant of formula (I) wherein G comprises a group G′ representing a group of formula (XXIV) wherein A₁₀₁ represents O then being formed,
G₁ represents a hydroxyl or an amine —NH₂ and G₂ represents a group of the following formula

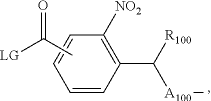

a surfactant of formula (I) wherein G comprises a group G′ representing a group of formula (XIXIV) wherein A₁₀₁ respectively represents —O—(CO)— or —NH—(CO) then being formed,
G₁ represents a good leaving group LG and G₂ represents a hydroxyl, a surfactant of formula (I) wherein G comprises a group G′ representing a group of formula (XXV) then being formed,
G₁ represents a good leaving group LG and G₂ represents an amine —NH₂, a surfactant of formula (I) wherein G comprises a group G′ representing a group of formula (XXVI) then being formed,
G₁ represents an oxyamine —O—NH₂ and G₂ represents an aldehyde, a surfactant of formula (I) wherein G comprises a group G′ representing a group of formula (XXVII) then being formed.

When the group G comprises several groups G′, the selection of the groups reacting together G′₁ and G₁ on the one hand, G′₂ and G₂ on the other hand, may be made in the same way, by replacing the groups G₁ or G₂ in the examples mentioned above with G′₁ or G′₂.

The emulsions 1 and 2 applied in the method may be prepared with the first aforementioned method comprising the steps (i), (ii), (iii) and (iv).

The invention will be described in more detail by means of the appended figure and of the examples which follow.

### FIGURE

FIG. 1 represents a diagram illustrating the positioning of the surfactant of formula (I) wherein v and w represent 1, and of the droplets of the formulation.

FIG. 2 represents a diagram illustrating the positioning of the surfactant of formula (I), wherein v and w represent 2, and of the droplets of the formulation.

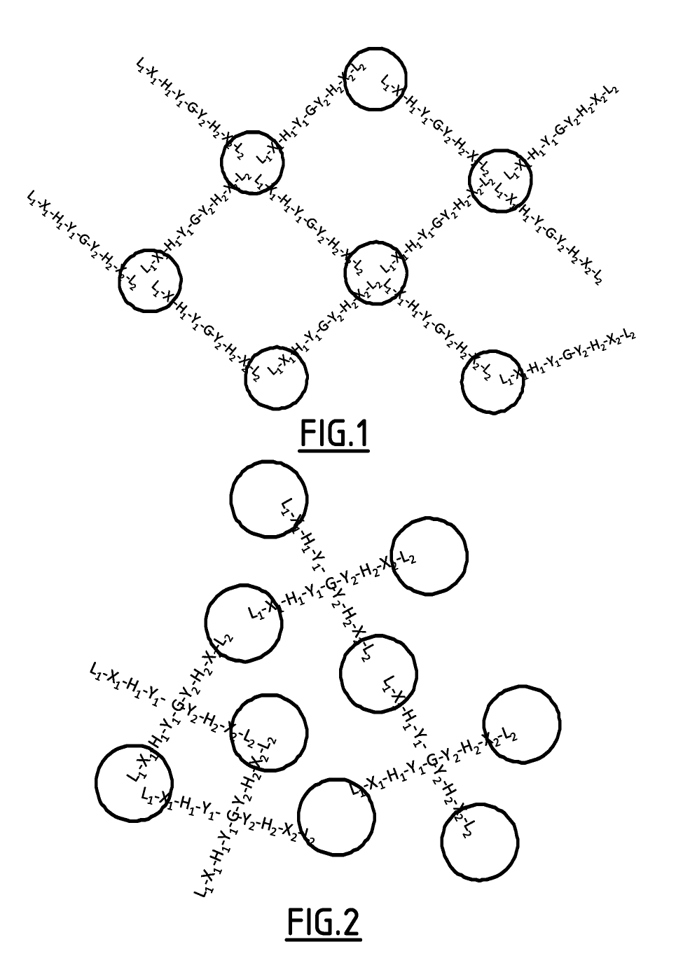

### EXAMPLES

### Example

### The Use of a Formulation Comprising Estrogen (Estradiol) or an Estrogen Agonist (Ethynil Estradiol)

Preparation and Composition of the Formulations

Six different formulations A, B, C, D, E and F were made, the compositions of which are indicated in Table 2.

TABLE 1
Composition of the formulations

| | | | Amount (for a 2 ml batch) | | | | | | % by mass of the | |
|---|---|---|---|---|---|---|---|---|---|---|
| | Raw material | Provider | A | B | C | D | E | F | total formulation | |
| Oily premix | Lipoid (mg) | Lipoid (mg) | 17 | | | | | | 0.85 | 5.4 |
| | Suppocire ® NC | Gattefossé | 68 | 68 | 68 | 68 | 34 | 68 | 3.4 | |
| | (mg) | | | | | | | | | |
| | Dynasan 114 | Sigma | — | — | — | — | 34 | — | | |
| | | Aldrich | | | | | | | | |
| | Soya bean oil | Sigma | 23 | | | | | | 1.15 | |
| | | Aldrich | | | | | | | | |
| | Estradiol | Sigma | 1 | 2 | — | — | — | — | 0 or | |
| | (mg) | Aldrich | | | | | | | 0.05 or 0.1 | |
| | Ethynil Estradiol | Sigma | — | — | 1 | 2 | 2 | 2 | 0 or | |
| | (mg) | Aldrich | | | | | | | 0.05 or 0.1 | |
| Aqueous phase | Myrj ® s40 | Croda | 92 | | | | | | 4.6 | — |
| | (mg) | | | | | | | | | |
| | PBS 1X | Sigma | 1,800 | | | | | 1,800 | 90 | — |
| | (μL) | Aldrich | | | | | | | | |

The proportion of dispersed phase in the tested formulations (ratio of the mass of [oily premix+co-surfactant PEG] over the mass of the whole of the emulsion) is of about 10%.

The following preparation method was followed:

(i) Preparation of the Oily Phase:

The soya bean oil, the Suppocire NC, optionally Dynasan 114, the lecithin, were weighed and then mixed with dichloromethane before being heated to 60° C. in order to obtain a homogeneous viscous solution. With dichloromethane it is possible to promote solubilization. The estradiol or ethynil estradiol was dissolved in ethanol and then the volume corresponding to the desired dose was incorporated into the aforementioned homogeneous viscous solution. The solvents are then evaporated in vacuo.

(ii) Preparation of the Aqueous Phase:

During the phase for evaporating the ethanol, the aqueous phase was prepared. In a 5 ml Eppendorf, the co-surfactant, glycerol and the aqueous PBS solution were mixed and then dissolved in a bath at 75° C.

(iii) Mixing Both Phases:

The oily phase is at about 40° C. (in a viscous form) and the aqueous phase is at about 70° C. (upon leaving the bath). The aqueous phase was poured into the oily phase.

(iv) Emulsification:

The flask containing both phases was attached in the sonication enclosure. The base of the flask (about 1 cm) was positioned in a water bath at 15° C. The conical sonotrode is immersed in the flask, the latter being placed at half-height (over about 1 cm in the solution) for better homogeneity in making the nanoemulsion. Sonication was then carried out with an AV505 Sonicator (Sonics, Newtown USA). The effective sonication time was 10 mins.

(v) Purification:

Purification was carried out by dialysis in a large volume of PBS 1× overnight. Glycerol is removed during this step. The purified nanoemulsion sample was sterilized by filtration on a 0.22 μm filter.

Size and Polydispersity Index of the Droplets of the Formulations

The size of the droplets of the formulations was measured by quasi-elastic scattering of light in a ZetaSizer, Malvern, in a solution of PBS 0.1× (Table 2).

TABLE 2
Size of the droplets and the polydispersity index
(PDI) measured by quasi-elastic light scattering

| | size (nm) | PDI |
|---|---|---|
| Formulation A | 52.63 | 0.188 |
| Formulation B | 54.9 | 0.175 |
| Formulation C | 56.09 | 0.188 |
| Formulation D | 52.11 | 0.161 |
| Formulation E | 63.04 | 0.172 |
| Formulation F | 51.13 | 0.115 |

Experiments were reproduced with emulsions kept for 30 days at 4° C. after dialysis (Table 3). The results of Table 3 show that the sizes and PDIs are the same after 30 days, which shows the stability of the emulsions over time.

TABLE 3
Size of the droplets and the polydispersity index (PDI) measured by quasi-elastic
light scattering of the formulations after dialysis or 30 days after dialysis.

| | Just after dialysis | | | | 30 days after dialysis | | | |
|---|---|---|---|---|---|---|---|---|
| | size (nm) | | PDI | | size (nm) | | PDI | |
| Formulation A | 52.63 | 1.056 | 0.188 | 0.00376 | 53 | 1.063 | 0.2 | 0.011 |
| Formulation B | 54.9 | 1.098 | 0.175 | 0.0035 | 55.94 | 0.9351 | 0.19 | 0.014 |
| Formulation C | 56.09 | 1.1218 | 0.188 | 0.00376 | 57.52 | 0.8764 | 0.19 | 0.016 |
| Formulation D | 42.11 | 0.8422 | 0.161 | 0.00322 | 42.03 | 0.44 | 0.15 | 0.003 |
| Formulation E | 63.04 | 1.2608 | 0.172 | 0.00344 | 64.11 | 1.167 | 0.18 | 0.006 |
| Formulation F | 51.13 | 1.0226 | 0.115 | 0.0023 | 51.04 | 1.161 | 0.15 | 0.0024 |

The right columns correspond to the standard deviation for three series of 15 measurements (i.e. a total of 45 measurements).

Zeta Potential of the Droplets of the Formulations

The zeta potential of the droplets of the formulations was measured with a zetasizer, Malvern in PBS 0.1× (Table 4).

TABLE 4
Zeta potential of the formulations

| | Zeta (mV) |
|---|---|
| Formulation A | −2.71 |
| Formulation B | −4.14 |
| Formulation C | −1.98 |
| Formulation D | −4.89 |
| Formulation E | −6.55 |
| Formulation F | −6.8 |

HPLC Assay of the Amount of Estradiol or Ethynil Estradiol Encapsulated in the Droplets

The amount of estradiol or ethynil estradiol encapsulated in each formulation is assayed by HPLC (an assay method from the European Pharmacopeia with detection by UV absorption at 230 and 286 nm (analytical HPLC, Water Alliance. Room temperature (25° C.), 60% acetonitrile and 50% water. A reverse phase C18 column).

The encapsulation and leaking levels of estradiol or of ethynil estradiol were evaluated by HPLC by assaying the estradiol or ethynil estradiol before dialysis (reference 100%), after dialysis (with which it is possible to infer the encapsulation level) and in the dialysis bath (possible burst release), by using standard curves (in acetonitrile) produced beforehand. The results are grouped in Table 5.

TABLE 5
Encapsulation percentage of estradiol
and ethynil estradiol after dialysis

Encapsulation %

| | A(286 nm) | A(230 nm) |
|---|---|---|
| Formulation A | 102 | 103 |
| Formulation B | 81 | 86 |
| Formulation C | 96 | 103 |
| Formulation D | 75 | 85 |
| Formulation E | 87 | 86 |
| Formulation F | 100 | 100 |

These results show that estradiol or ethynil estradiol do not leak out of the droplets after encapsulation (encapsulation % between 85% and 105% according to the assay, with an error of +/−10%, taking into account the extraction method).

The experiments were reproduced with emulsions kept for 30 days at 4° C. after dialysis (Table 6). The comparison of the data from Tables 4 and 5 shows that no leak of estradiol or ethynil estradiol occurred, which shows the stability of the emulsions over time.

TABLE 6
Encapsulation percentage of estradiol or
ethnyl estradiol, 30 days after dialysis

Encapsulation %

| | A(286 nm) | A(230 nm) |
|---|---|---|
| Formulation A | 116 | 85 |
| Formulation B | 95 | 96 |
| Formulation C | 110 | 117 |
| Formulation D | 108 | 103 |
| Formulation E | 106 | 118 |
| Formulation F | 101 | 107 |

1. A hormonal treatment method comprising administration in a mammal in need thereof, of an effective amount of the formulation as a nanoemulsion, comprising a continuous aqueous phase and at least one dispersed phase and comprising:
an amphiphilic lipid,
a solubilizing lipid comprising at least one glyceride of fatty acids,
a hormone, a hormone agonist or antagonist or a mixture thereof, and
a co-surfactant comprising at least one chain consisting of alkylene oxide units,
wherein the hormonal treatment is selected from post-menopausal hormonal treatment, contraception, reproduction assistance, treatment of menorrhagias, treatment of acne, and treatment of adrenal dysregulations such as Addison's disease, adrenal fatigue and infertility.

2. The method according to claim 1, wherein:
the hormone is selected from:
estrogen, notably selected from estrone estriol and estradiol,
progesterone
hydrocortisone
5-dehydroepiandrosterone
gonadotropins,
kisspeptin,
the hormone agonist is:
an estrogen agonist, such as tibolone or an estrogen derivative selected from estrone, estril and estradiol esters, estropipate, estrodiol cypionate, conjugate equine estrogens and ethinyl estradiol, or
a progestin (agonist of progesterone), such as medroxyprogesterone acetate, norethisterone, norethisterone acetate, lynestenol, levonorgestrel, norgestrel, norgestimate, desogestrel, etonogestrel, gestodene, nogestimate, norelgestromine, nandralone, 17-hydroxyprogesterone or drospirenone,
the hormone antagonist is:
an antiandrogen, such as cyproterone acetate, or
an antagonist of estrogens, such as clomifene citrate, tamoxifene, raloxifene, toremifene or lasofoxifene.

3. The method according to claim 1, wherein the selected hormone is:
for contraception:
estrogen or an estrogen agonist, preferably ethinyl estradiol, and/or
progesterone or a progestin, or
for treatment of acne:
an antiandrogen, such as cyproterone acetate, and/or
a progestin, such as medroxyprogesterone acetate, drospirenone or norgestimate, or
for treatment of menorrhagias: progesterone or a progestin, or
for treatment of infertility due to adrenal dysregulation or for assistance with impregnation, tamoxifene, clomifene citrate, gonadotropins or kisspeptin, or
for treating Addison's disease: hydrocortisone, or
for treatment of adrenal fatigue, hydrocortisone or 5-dehydroepiandrosterone, and optionally pantothenic acid and/or phosphatidylserine, or
for post-menopausal hormonal treatment:
estrogen or an estrogen antagonist, such as tibolone or an estrogen derivative selected from estrone, estril and estradiol esters, estropipate, estradiol cypionate, conjugate equine estrogen and ethinyl estradiol, preferably ethinynl estradiol, and
optionally progesterone or a progestin.

4. The method according to claim 1, wherein the amphiphilic lipid is a phospholipid.

5. The method according to claim 1, wherein the solubilizing lipid consists in a mixture of glycerides of saturated fatty acids including at least 10% by weight of C12 fatty acids, at least 5% by weight of C14 fatty acids, at least 5% by weight of C16 fatty acids and at least 5% by weight of C18 fatty acids.

6. The method according to claim 1, wherein the oily phase comprises at least one oil.

7. The method according to claim 1, wherein the co-surfactant includes at least one chain consisting of ethylene oxide units or ethylene oxide and propylene oxide units.

8. The method according to claim 1, comprising an imaging agent.

9. The method according to claim 1, wherein the dispersed phase represents between 35 and 65% by weight based on the total weight of the nanoemulsion.

10. The method according to claim 1, comprising a surfactant of the following formula (I):
(L₁-X₁—H₁—Y₁)ᵥ-G-(Y₂—H₂—X₂-L₂)_{w} (I),
wherein:
L₁ and L₂ represent independently lipophilic groups,
X₁, X₂, Y₁, Y₂ and G represent independently a binding group,
H₁ and H₂ represent independently hydrophilic groups comprising a polyalkoxylated chain,
v and w are independently an integer from 1 to 8,
and wherein the droplets of the dispersed phase are bound together covalently with the surfactant of formula (I).
